(19) United States
(12) Patent Application Publication
(10) Pub. No.: US 2006/0293656 A1
(43) Pub. Date: Dec. 28, 2006

(54) Electrosurgical instrument and method of use

(76) Inventors: John H. Shadduck, (US); Csaba Truckai, Saratoga, CA (US)

Correspondence Address: Attn: John H. Shadduck;SurgRx Inc., 380 Portage Ave, Palo Alto, CA, 94306, US

(21) Appl. No.: 11/196,525

(22) Filed: Aug. 3, 2005

Related U.S. Application Data
(63) Continuation-in-part of application No. 10/032,867, filed on Oct. 22, 2001, now Pat. No. 6,929,644.
(63) Continuation-in-part of application No. 10/351,449, filed on Jan. 22, 2003, now Pat. No. 7,112,201.
(60) Provisional application No. 60/598,713, filed on Aug. 3, 2004.

(51) Int. Cl. A61B 18/14

(52) U.S. Cl. 606/51

(57) ABSTRACT

Electrosurgical jaw structures are disclosed that include pressure sensitive variable resistive materials in electrosurgical energy delivery surfaces for welding tissue. The pressure sensitive materials are configured to have megaohm impedance when not engaging tissue and can transform into highly conductive electrodes when compressed under a selected pressure. In a method of the invention, the pressure sensitive variable resistive materials prevent arcing and tissue desiccation when applying bi-polar Rf current to tissue engaged under high compression in an electrosurgical jaw structure.

### CROSS-REFERENCE TO RELATED APPLICATIONS

This application claims benefit of U.S. Provisional Patent Application Ser. No. 60/598,713 filed Aug. 3, 2004 titled Surface-Conforming Electrosurgical Electrode; and this application is a continuation-in-part of U.S. patent application Ser. No. 10/032,867 filed Oct. 22, 2001 titled Electrosurgical Jaw Structure for Controlled Energy Delivery, and this application is also a continuation-in-part of Ser. No. 10/351,449 filed Jan. 22, 2003 titled Electrosurgical Instrument and Method of Use; all of the above applications are incorporated herein and made a part of this specification by this reference.

### BACKGROUND OF THE INVENTION

Field of the Invention

Embodiments of the invention relate to medical devices and methods and more particularly relates to an electrosurgical jaw structure and methods for creating high strength welds in tissue.

In the prior art, various energy sources such as radiofrequency (Rf) sources, ultrasound sources and lasers have been developed to coagulate, seal or join together tissues volumes in open and laparoscopic surgeries. One surgical application relates to sealing blood vessels which contain considerable fluid pressure therein. In general, no instrument working ends using any energy source have proven reliable in creating a “tissue weld” or “tissue fusion” that has very high strength immediately post-treatment. For this reason, the commercially available instruments, typically powered by Rf or ultrasound, are mostly limited to use in sealing small blood vessels and tissues masses with microvasculature therein. The prior art Rf devices also fail to provide seals with substantial strength in anatomic structures having walls with irregular or thick fibrous content, in bundles of disparate anatomic structures, in substantially thick anatomic structures, or in tissues with thick fascia layers (e.g., large diameter blood vessels).

The effect of RF waves was first reported by d'Arsonval in 1891. (see d'Arsonval, M. A., Action physiologique des courants alternatifs; CR Soc Biol.; 1891; 43:283-286). He described heating of tissue when the RF waves pass through living tissue. This led to the development of medical diathermy. The physical principles of tissue interaction with Rf waves was first described by Organ, who demonstrated that alternating current causes agitation of ions in the living tissue that results in frictional heat and thermal effects (see Organ, L. W., Electrophysiologic principles of radiofrequency lesion making. Appl Neurophysiol.; 1976; 39:69-76). A typical Rf system consists of a very high frequency (200 to 1200 KHz) alternating current generator, an Rf monopolar electrode and ground pad (a large dispersive electrode) or a bi-polar electrode arrangement, with the electrodes and targeted tissue all connected in series. In such a circuit, Rf current enters through both the electrodes with the engaged tissue functioning as a resistor component. As the Rf current alternates in directions at high frequency, tissue ions that are attempting to follow the direction of the current are agitated. Due to natural high resistivity in the living tissue, ionic agitation produces frictional heat between bi-polar electrodes in a working end. In a mono-polar electrode, because the grounding pad has a very large surface area, the electrical resistance is low at the ground pad and hence the ionic frictional heat is concentrated at the mono-polar electrode.

Thus, the application of electromagnetic energy from Rf current produces thermal effects, the extent of which is dependent on temperature and Rf application duration. At a targeted temperature range between about 70° C. and 90° C., there occurs heat-induced denaturation of proteins. At any temperature above about 100° C., the tissue will vaporize and tissue carbonization can result.

In a basic jaw structure with a bi-polar electrode arrangement, each face of opposing first and second jaws comprises an electrode and Rf current flows across the captured tissue between the opposing polarity electrodes. Such prior art Rf jaws that engage opposing sides of tissue typically cannot cause uniform thermal effects in the tissue-whether the captured tissue is thin or substantially thick. As Rf energy density in tissue increases, the tissue surface becomes desiccated and resistant to additional ohmic heating. Localized tissue desiccation and charring can occur almost instantly as tissue impedance rises, which then can result in a non-uniform seal in the tissue. The typical prior art Rf jaws can cause further undesirable effects by propagating Rf density laterally from the engaged tissue thus causing unwanted collateral thermal damage.

The commercially available Rf sealing instruments typically adopt a “power adjustment” approach to attempt to control Rf flux in tissue wherein a system controller rapidly adjusts the level of total power delivered to the jaws' electrodes in response to feedback circuitry coupled to the electrodes that measures tissue impedance or electrode temperature. Another approach used in the prior art consists of jaws designs that provide spaced apart of offset electrodes wherein the opposing polarity electrode portion s are spaced apart by an insulator material—which may cause current to flow within an extended path through captured tissue rather that simply between opposing electrode surfaces of the first and second jaws. Electrosurgical grasping instruments having jaws with electrically-isolated electrode arrangements in cooperating jaws faces were proposed by Yates et al. in U.S. Pat. Nos. 5,403,312; 5,735,848 and 5,833,690. In general, the prior art instruments cannot reliably create high strength seals in larger arteries and veins.

### BRIEF SUMMARY OF THE INVENTION

Various embodiments of the invention provide electrosurgical instrument systems assemblies and methods that utilize a novel means for modulating Rf energy application to biological tissue to create high strength thermally welds or seals in targeted tissues. In some embodiments, the system is configured to allow for a “one-step” welding-transecting procedure wherein the surgeon can contemporaneously (i) engage tissue within a jaw structure (ii) apply Rf energy to the tissue, and (iii) transect the tissue. Particular embodiments also provide systems and methods for Rf welding of tissue with a reduction or elimination of arcing and tissue desiccation.

Various embodiments also provide a jaw structure that can engage and weld tissue bundles, defined herein as bundles of disparate tissue types (e.g., fat, blood vessels, fascia, etc.). For the welding of tissue bundles, it is desirable that the jaw surfaces apply differential energy levels to each different tissue type simultaneously. Accordingly, embodiments of the invention provide an electrosurgical system that is configured to apply differential energy levels across the jaws engagement surfaces with “smart” materials without the need for complex feedback circuitry coupled to thermocouples or other sensors in the jaw structure. These and related embodiments allow for contemporaneously modulation of energy densities across the various types of in the tissue bundle according to the impedance of each engaged tissue type and region.

In order to create the most effective “weld” in tissue, it is desirable that the targeted volume of tissue be uniformly elevated to the temperature needed to denature proteins therein. To create a “weld” in tissue, collagen and other protein molecules within an engaged tissue volume are desirably denatured by breaking the inter- and intra-molecular hydrogen bonds—followed by re-crosslinking on thermal relaxation to create a fused-together tissue mass. It can be easily understood that ohmic heating in tissue—if not uniform—can at best create localized spots of truly “welded” tissue. Such a non-uniformly denatured tissue volume still is “coagulated” and will prevent blood flow in small vasculature that contains little pressure. However, such non-uniformly denatured tissue will not create a seal with significant strength, for example in 2 mm. to 10 mm. arteries that contain high pressures.

Various embodiments of systems and methods of the invention relate to creating thermal “welds” or “fusion” within native tissue volumes. The alternative terms of tissue “welding” and tissue “fusion” are used interchangeably herein to describe thermal treatments of a targeted tissue volume that result in a substantially uniform fused-together tissue mass, for example in welding blood vessels that exhibit substantial burst strength immediately post-treatment. The strength of such welds is particularly useful (i) for permanently sealing blood vessels in vessel transection procedures, (ii) for welding organ margins in resection procedures, (iii) for welding other anatomic ducts wherein permanent closure is required, and also (iv) for vessel anastomosis, vessel closure or other procedures that join together anatomic structures or portions thereof. The welding or fusion of tissue as disclosed herein is to be distinguished from “coagulation”, “sealing”, “hemostasis” and other similar descriptive terms that generally relate to the collapse and occlusion of blood flow within small blood vessels or vascularized tissue. For example, any surface application of thermal energy can cause coagulation or hemostasis—but does not fall into the category of “welding” as the term is used herein. Such surface coagulation does not create a weld that provides any substantial strength in the affected tissue.

At the molecular level, the phenomena of truly “welding” tissue as disclosed herein may not be fully understood. However, the authors have identified the parameters at which tissue welding can be accomplished. An effective “weld” as disclosed herein results from the thermally-induced denaturation of collagen, elastin and other protein molecules in a targeted tissue volume to create a transient liquid or gel-like proteinaceous amalgam. A selected energy density is provided in the targeted tissue to cause hydrothermal breakdown of intra- and intermolecular hydrogen crosslinks in collagen and other proteins. The denatured amalgam is maintained at a selected level of hydration—without desiccation—for a selected time interval which can be very brief. The targeted tissue volume is maintained under a selected very high level of mechanical compression to insure that the unwound strands of the denatured proteins are in close proximity to allow their intertwining and entanglement. Upon thermal relaxation, the intermixed amalgam results in “protein entanglement” as re-crosslinking or renaturation occurs to thereby cause a uniform fused-together mass.

Various embodiments of the invention provide an electrosurgical jaw structure comprising first and second opposing jaws wherein at least one jaw carries a pressure sensitive variable resistance material that deforms slightly under tissue-engaging pressure and can be transformed from an insulative layer to a conductive electrode layer under a selected pressure level. The pressure sensitive surface will thus adjust Rf current flow therethrough in response to local tissue-engaging pressure. The pressure sensitive variable resistance material thus can deliver high amount of energy to more highly compressed tissue, and limit electrosurgical energy delivery into desiccated tissue regions that shrink to prevent arcs and tissue charring.

### BRIEF DESCRIPTION OF THE DRAWINGS

FIG. 1 is a perspective view of an exemplary surgical instrument with first and second jaws, with at least one jaw having a pressure sensitive surface layer that increases conductance under tissue-engaging pressure.

FIG. 2A is an enlarged perspective view of the opposing jaws of FIG. 1 in an open position.

FIG. 2B is a view of the opposing jaws of FIG. 2 in a closed position.

FIG. 3 is a perspective view of the opposing jaws of FIG. 2A from a different angle showing the pressure sensitive surface layer in the upper jaw.

FIG. 4 is a perspective view of a forceps device with opposing jaws that both carry pressure sensitive surface layers.

FIG. 5 is a chart illustrating the pressure-resistance profile of an exemplary pressure sensitive material for electrosurgical jaw surfaces.

FIG. 6A is a sectional schematic view of a jaw structure as in FIG. 4 with pressure sensitive surfaces initially engaging tissue.

FIG. 6B is a sectional view as in FIG. 6A with the jaw structure applying high pressures to tissue wherein the pressure sensitive surfaces deform to adjust current flow therethrough.

FIG. 6C is a longitudinal sectional view of a jaw structure as in FIG. 6A illustrating the prevention of edge effects such as arcing in tissue.

FIG. 7 is a sectional view of the jaw structure of FIG. 2B showing a pressure sensitive surface in a single jaw.

FIG. 8 is a sectional view of a jaw structure wherein the pressure sensitive materials are interior of the jaw surfaces.

FIG. 9 is a sectional view of a jaw structure that includes a pressure sensitive material and an auxetic material for causing enhanced local tissue compression.

### DETAILED DESCRIPTION OF THE INVENTION

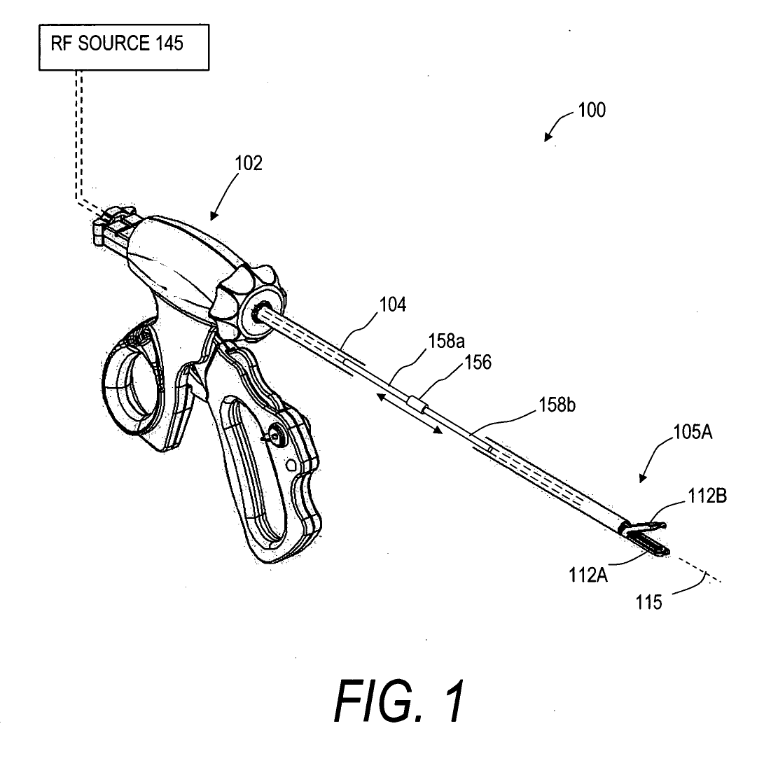

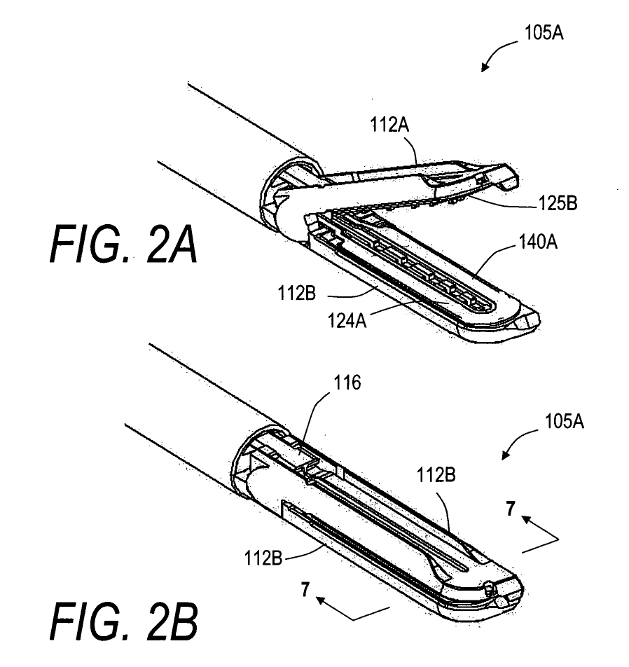

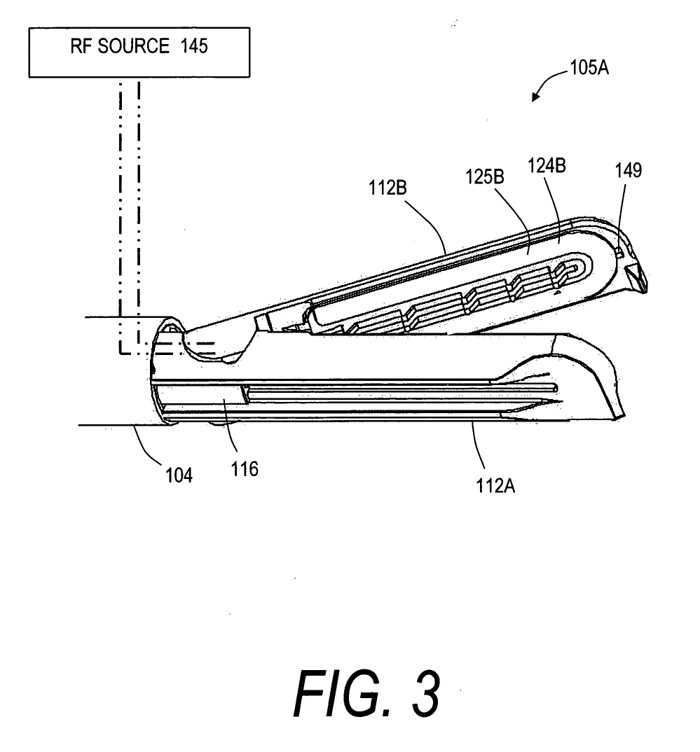

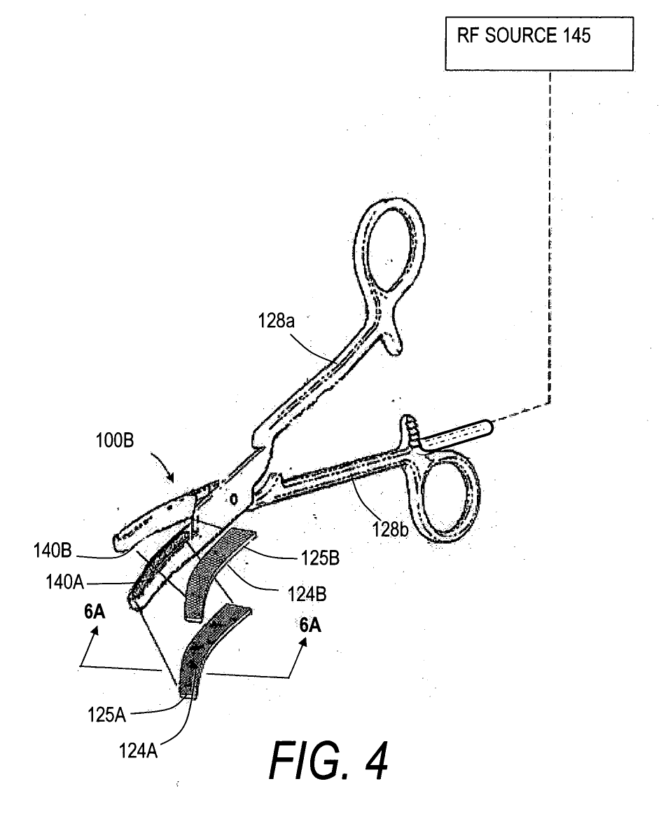

FIG. 1 illustrates an exemplary instrument 100A having handle 102 that is coupled to introducer member 104 that carries a working end comprising an electrosurgical jaw structure 105A corresponding to the invention. The jaw structure includes first (lower) jaw element 112A and second (upper) jaw element 112B that close or approximate about axis 115. The tissue-engaging surfaces 124A and 124B of jaws 112A and 112B carry electrosurgical functionality for sealing or welding tissue. In one embodiment as in FIGS. 2A-2B and 3, at least one jaw carries (the upper jaw) carries a surface layer 125B of a pressure sensitive variable resistive material for controlling bi-polar Rf energy delivery to engaged tissue. Any electrosurgical jaw structure can carry such pressure sensitive surfaces, which includes endoscopic and open surgery instruments with any curved or straight jaw shapes. The jaws can be opened and closed by any suitable mechanism. In one embodiment shown in FIGS. 1-3, the jaws include a slidable cutting blade in the form of a transverse I-beam member 126 that also is configured as a jaw closing mechanism, and is described in more detail in co-pending U.S. Pat. Appl. Ser. No. 10/351,449 filed Jan. 22, 2003. In FIG. 4, a forceps device for open surgery is shown with jaw structure 100B that is configured with pressure sensitive electrosurgical surfaces 125A and 125B in both jaw's tissue-engaging surfaces. The forceps of FIG. 4 has opposing polarity electrodes in the opposing jaws, with electrical leads in handles 128a and 128b as in known in the art.

In one embodiment, the pressure sensitive material 125A or 125B comprises a non-conductive polymer that is doped with conductive elements or particles, as generally described in co-pending U.S. patent application Ser. No. 10/351,449 filed Jan. 22, 2003 titled Electrosurgical Instrument and Method of Use; Ser. No. 10/032,867 filed Oct. 22, 2001 titled Electrosurgical Jaw Structure for Controlled Energy Delivery; and Ser. No. 10/308,362 filed Dec. 3, 2002 (now U.S. Pat. No. 6,770,072), which are incorporated herein by reference and are made a part of this specification. In one embodiment, the pressure sensitive material is a medical grade silicone polymer that is doped with conductive particles or granules such as carbon or a metal. The metal can include at least one of titanium, tantalum, stainless steel, silver, gold, platinum, nickel, tin, nickel titanium alloy, palladium, magnesium, iron, molybdenum, tungsten, zirconium, zinc, cobalt or chromium and alloys thereof. The metal or carbon can be in the form of at least one of particles, granules, grains, flakes, microspheres, spheres, powders, filaments, crystals, rods, nanotubes and the like. The mean dimension of the conductive particles or granules can range from about 1 micron to 250 microns, and more preferably from about 5 microns to 100 microns.

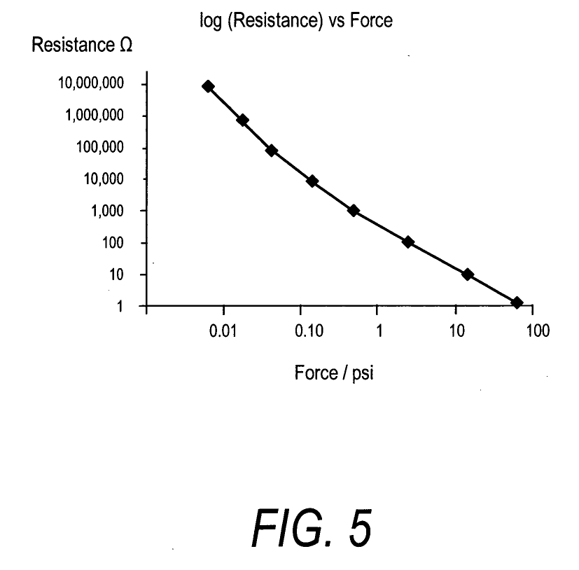

FIG. 5 is a chart illustrating the pressure-resistance profile of an exemplary pressure sensitive variable resistance material suitable for at least one jaw surface in instruments as in FIGS. 1-4. The chart indicates that resistance can be in the megaohm range in a first repose or quiescent insulative state. Under a selected level of tissue-engaging pressure, the resistance can be reduced even to a milliohm range to provide its second conductive state. In one embodiment, the material in a first insulative state has an impedance of greater than 1,000 ohms/cm, or greater than 10,000 ohms/cm, or greater than 100,000 ohms/cm. In one embodiment, the material in a second conductive state has an impedance of less than 500 ohms/cm; or less than 50 ohms/cm; of less than 5 ohms. The pressure required transform the material from the first substantially insulative state to the second substantially conductive state can be within a range suitable for welding tissue, and can range between 0.5 psi and 500 psi; or between 5 psi and 250 psi. Pressure sensitive resistive materials are disclosed in U.S. Pat. No. 4,028,276 to Harden, et al; in U.S. Pat. No. 4,120,828 to Michalchik; and in U.S. Pat. No. 6,291,568 to Lussey, all of which patents are incorporated herein by this reference.

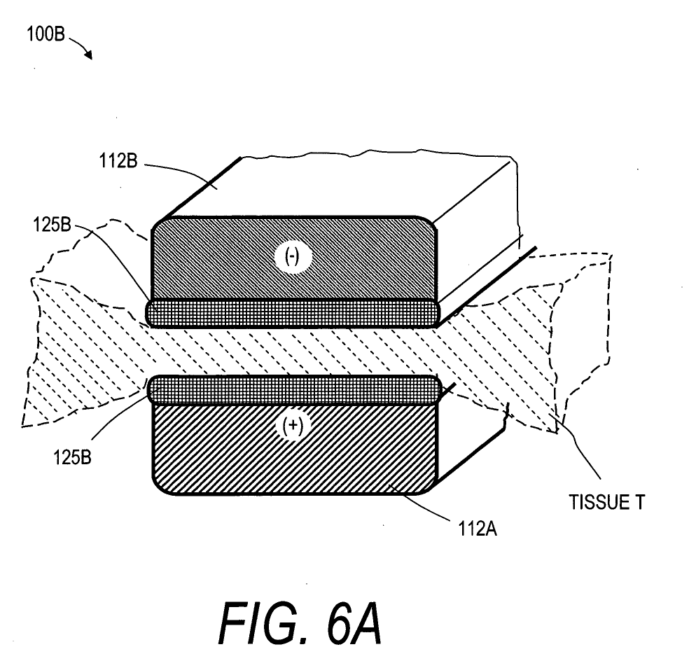

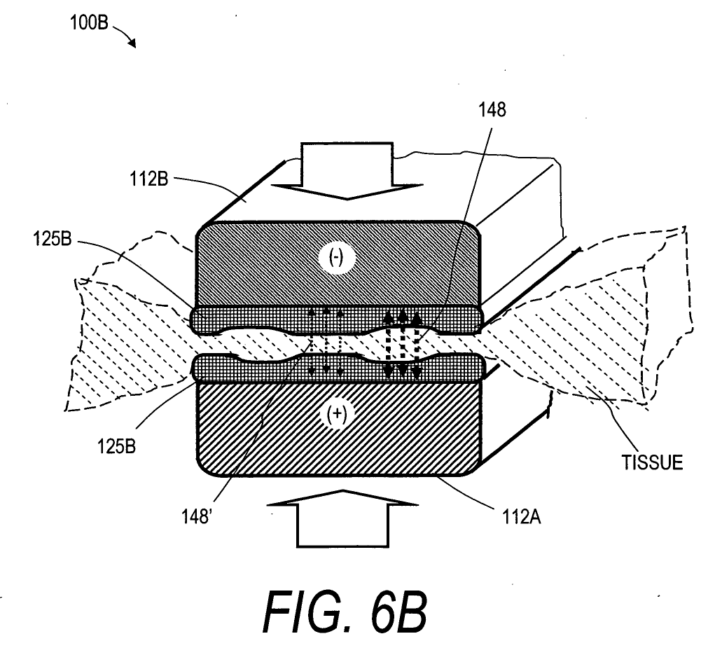

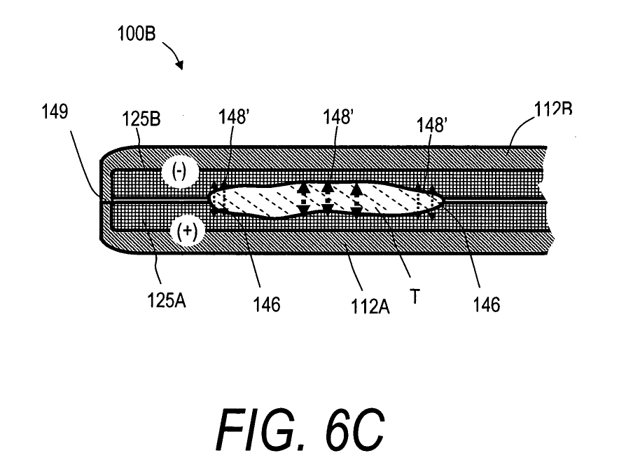

Now turning to FIGS. 6A-6B, an electrosurgical method of the invention is shown wherein the pressure sensitive resistive material is configured for controlling Rf current flows in tissue to thereby control the resultant ohmic tissue heating. The schematic jaw structure in FIGS. 6A-6B corresponds to the forceps jaws of FIG. 4, wherein both tissue-engaging surfaces 124A and 124B of jaws 112A and 112B carry a pressure-sensitive body 125A or 125B for controlling bi-polar Rf energy. In FIG. 6A, it can be seen that pressure sensitive material 125A comprises a surface layer that overlies a first polarity (+) conductor 140A that is connected to Rf source 145. Similarly, pressure sensitive material 125B comprises a surface layer in upper jaw 112B that is coupled to second (−) polarity conductor 140B that also is connected to Rf source 145. In this embodiment, the structural components of the jaws can be any suitable electrically conductive material such as stainless steel, that also function as the first and second polarity conductors which are insulated from one another as is known in the art.

In FIG. 6A, the engagement surfaces are in a quiescent, planar form when beginning to engage tissue T under minimal compression, for example, with forces under about 1 psi. FIG. 6B next illustrates further jaw closure wherein the engagement surfaces apply very high compression to the tissue, for example more than 5 psi and even more than 250 psi. Under the selected tissue-engaging pressure, the jaw surfaces will conform to the tissue wherein higher density tissue portions can more highly compress the pressure sensitive surfaces 125A and 125B. After the tissue T is compressed as in FIG. 6B, or contemporaneous with engaging the tissue, the physician actuates bi-polar Rf current delivery to the tissue. In one embodiment, all regions of surfaces 125A and 125B conduct Rf current therethrough under the engagement pressure. In another embodiment, the surfaces 125A and 125B conduct Rf current in proportion to the local tissue-engaging pressure, as indicated in FIG. 6B. Higher Rf current density occurs in region 148 and lower Rf current density occurs in region 148′. During operation, the desiccation of tissue can locally or regionally which thereby reduce the tissue cross-section. The pressure sensitive material then can adjust locally to the reduced pressure and dynamically adjust Rf current paths and energy density in the engaged tissue. It can be understood from FIG. 6B that Rf current paths can provide initial rapid ohmic heating in regions of highest tissue compression. Further, the method of the invention adjusts Rf current paths to modulate ohmic heating in engaged tissue as its conductive parameters (impedance, temperature, and hydration) dynamically change during Rf energy application. Of particular interest, the pressure sensitive surfaces 125A and 125B alter current flow paths to eliminate arcing and tissue desiccation since currents are re-directed away from desiccated tissue regions that tend to apply less pressure against the jaw surfaces. The pressure sensitive surfaces are particularly useful in opposing jaws to prevent edge effects such arcing, tissue desiccation and charring around the edges of tissue engaged in the jaws as shown in shown the schematic longitudinal jaw section of FIG. 6C. It can be seen that the highest Rf current density 148 will occur where the pressure sensitive surfaces 125A and 125B are most compressed. At the edges 146 of the tissue, a lower Rf current density 148′ will occur because of less compression of surfaces 125A and 125B. A periphery of the structural component of the jaw indicated at 149 in FIG. 6A and FIG. 3 serves as a stop to prevent the pressure sensitive surfaces 125A and 125B from contacting one another under substantial pressure to thereby prevent direct shorting of current between the jaw surfaces. In FIG. 6C, it thus can be seen that current density will be very low at the edges 146 of the tissue which will prevent arcs from jumping between the surfaces 125A and 125B about the tissue edges 146.

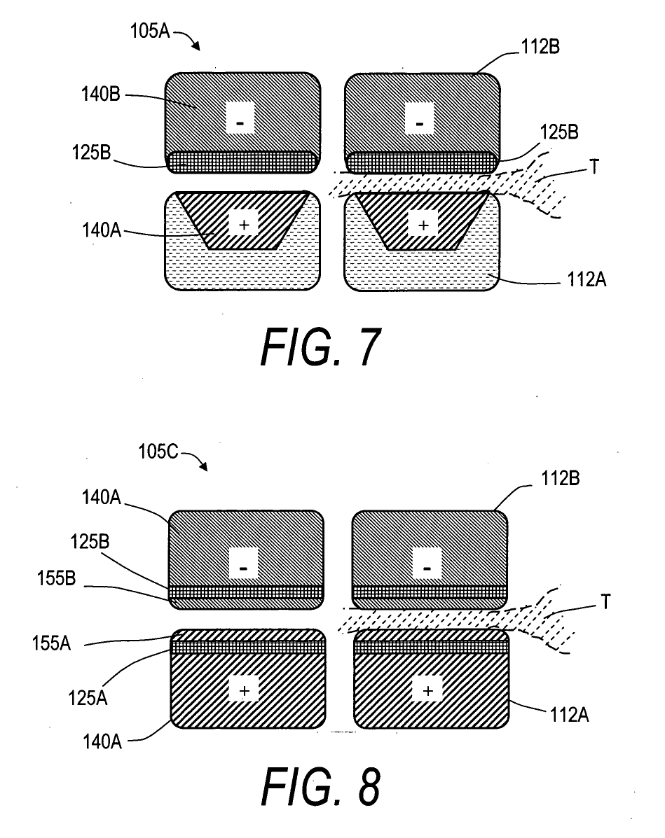

FIG. 7 is a sectional view of jaw structure 100A of FIGS. 1-3 wherein only one jaw surface 124B carries a pressure sensitive surface 125B. The lower jaw 112A has a tissue-engaging surface that comprises first polarity conductor 140A. This embodiment would function in a manner similar to that depicted in FIG. 6B above. FIG. 8 illustrates another embodiment of jaw structure 105C similar to that of FIGS. 1-3 wherein the jaws carry pressure sensitive variable resistive bodies 125A and 125B. In this embodiment, the pressure sensitive layers 150A, 150B are disposed in an interior of the jaws with conductors 155A and 155B comprising the tissue-engaging surfaces. The surface layers can be thin or thick members having either flexible or rigid properties. In use, tissue-engaging pressure would then determine the level of Rf current flowing from electrodes 140A and 140B through the pressure sensitive layers 150A, 150B to surfaces 155A and 155B and the tissue.

In a related embodiment, referring back to FIG. 1, the pressure sensitive system for controlling Rf energy delivery also can comprise a pressure sensitive variable resistive link 156 in a jaw closing mechanism. For example, in FIG. 1, a reciprocatable shaft that translates to close the jaws which comprises first member 158a and second member 158b together with pressure sensitive variable resistive link 156 coupled between the two shaft portions. A current path goes through the pressure sensitive link 156 to the electrosurgical surfaces to adjust current flow based on pressure being applied on the shaft to close the jaws.

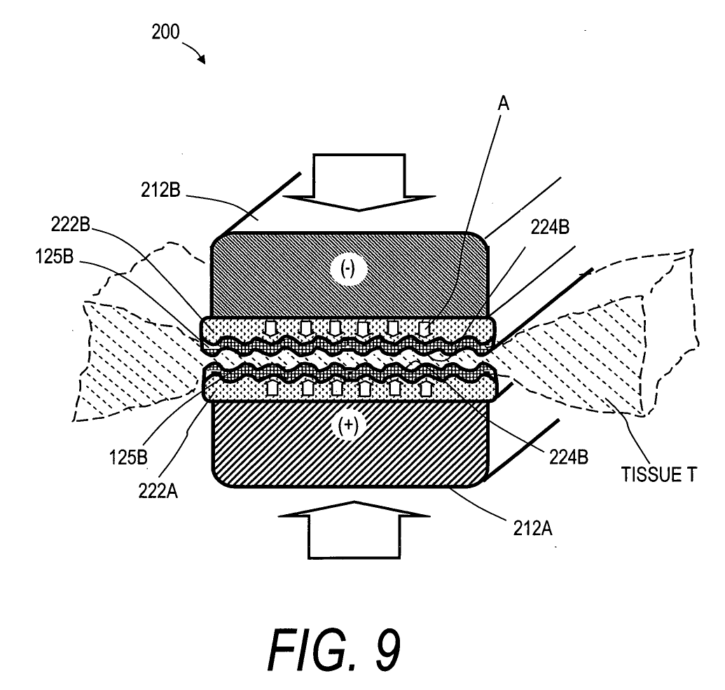

FIG. 9 illustrates another forceps jaw 200 in sectional view with opposing jaws 212A and 212B for engaging tissue, wherein the complaint tissue-engaging surfaces 224A and 224B have novel properties for engaging and conforming to non-uniform tissue surfaces. In FIG. 9, the jaw surfaces again can include pressure sensitive variable resistive layers 125A and 125B as described above. Another layer in the jaw comprises an elastomer material that provides novel and counterintuitive responses to tissue-compressing forces to enhance the jaw surface contact with tissue. In one embodiment, the jaws carry a layer of an auxetic polymeric material indicated at 222A and 222B that is coupled to the flexible electrosurgical energy delivery surfaces. An auxetic material has unique characteristics in that, when stretched lengthways, the material gets fatter rather than thinner in cross section. This characteristic can be used in a compliant electrosurgical surface so that when tissue is engaged under high pressure, the surface layer will tend to be displaced or stretched laterally—which in turn will cause transverse (vertical) expansion of the auxetic material (see arrows A in FIG. 9) in any regions wherein the auxetic polymer is adjacent less dense tissue. It can be understood that an auxetic material can optimize contact between the electrosurgical surfaces and the tissue to optimize and modulate Rf energy delivery to the tissue for preventing tissue desiccation, charring and arcing. In the embodiment of FIG. 9, the auxetic material may be conductively doped to transmit Rf current through the material to the surface, or the pressure sensitive surface layer 125A, 125B may have a direct connection with a Rf generator 145 wherein the auxetic material is configured only for applying forces on the surface layers.

Auxetic behavior in a polymer is also defined as a property that reflects a negative Poisson's ratio. Poisson's ratio is defined as the ratio of the lateral contractile strain to the longitudinal tensile strain for a material undergoing uniaxial tension in the longitudinal direction. In other words, the Poisson's ratio determines how the thickness of the material changes when it is stretched axially or lengthways. For example, when an elastic band is stretched axially the rubber material becomes thinner, giving it a positive Poisson's ratio. Elastomeric materials and solids typically have a Poisson's ratio of around 0.2-0.4. Poisson's ratio is determined by the internal structure of the materials. Elasticity and hence auxetic behavior does not depend on scale. Elastic deformations can take place at domains ranging from the microscale to nanoscale (i.e., the molecular level). Within the molecular scale or domain, auxetic polymeric materials are known that have a node and fibril structure (see U.S. Patent Application No. 20030124279 by Sridharan et al, published Jul. 3, 2003, incorporated herein by reference). Thus, the scope of the invention encompasses these domains ranging from auxetic molecular materials to auxetic microfabricated structures.

The above described structures are elastically anisotropic—that is, they have a different Poisson's ratio depending on the direction in which they are stretched. The concepts underlying auxetic materials were first developed in isotropic auxetic foams by Roderic Lakes at the University of Wisconsin, Madison. Polymeric and metallic foams were made with Poisson's ratios as low as −0.7 and −0.8, respectively. Methods for scaling down honeycomb-like cellular structures include LIGA technology, laser stereolithography, molecular self-assembly, silicon surface micromachining techniques and nanomaterials fabrication processes. Auxetic two-dimensional cellular structures with cell dimensions of about 50 microns have been made by Ulrik Larsen et al. at the Technical University of Denmark. Three-dimensional microstructures consisting of two-dimensional conventional and auxetic honeycomb patterns on cylindrical substrates have been designed and fabricates by George Whitesides et al. at Harvard University (see Xu B., Arias F., Brittain S. T., Zhao X.-M., Grzybowski B., Torquato S., Whitesides G. M., “Making negative Poisson's ratio microstructures by soft lithography”, Advanced Materials, 1999, v. 11, No 14, pp. 1186-1189). Other background materials on auxetic materials are: Baughman, R, “Avoiding the shrink”, Nature, 425, 667, 16 Oct. (2003); Baughman, R, Dantas, S. Stafstrom, S., Zakhidov, A, Mitchell, T, Dubin, D., “Negative Poisson's ratios for extreme states of matter”, Science 288: 2018-2022, Jun. (2000); Lakes, R. S., “A broader view of membranes”, Nature, 414, 503-504, 29 Nov. (2001); and Lakes, R. S., “Lateral Deformations in Extreme Matter”, perspective, Science, 288, 1976, Jun. (2000). All the preceding references are incorporated herein by this reference.

It should be appreciated that the scope of the invention extends to the use of comforming auxetic electrodes in electrosurgical and other applications that are not coupled to a pressure sensitive variable resistive surfaces.

The foregoing description of various embodiments of the invention has been presented for purposes of illustration and description. It is not intended to limit the invention to the precise forms disclosed. Many modifications, variations and refinements will be apparent to practitioners skilled in the art. Further, the teachings of the invention have broad application in the electrosurgical and laparoscopic device fields as well as other fields which will be recognized by practitioners skilled in the art.

Elements, characteristics, or acts from one embodiment can be readily recombined or substituted with one or more elements, characteristics or acts from other embodiments to form numerous additional embodiments within the scope of the invention. Hence, the scope of the present invention is not limited to the specifics of the exemplary embodiment, but is instead limited solely by the appended claims.

What is claimed is:

1. A method of applying electrosurgical energy to tissue comprising,
providing an electrosurgical instrument having a jaw structure configured to engage tissue; and
applying electrosurgical energy from the jaws to engaged tissue wherein a pressure sensitive system connected to the instrument adjusts electrosurgical energy delivery in response to tissue-engaging pressure.

2. The method of applying electrosurgical energy to tissue of claim 1 wherein a pressure sensitive jaw surface adjusts electrosurgical energy delivery to tissue.

3. The method of applying electrosurgical energy to tissue of claim 2 wherein the pressure sensitive jaw surface adjusts electrosurgical energy delivery across the jaw surface in response to local tissue-engaging pressure.

4. The method of applying electrosurgical energy to tissue of claim 2 wherein a plurality of pressure sensitive jaw surface portions adjust electrosurgical energy delivery to adjacent engaged tissue in response to tissue-engaging pressure.

5. The method of applying electrosurgical energy to tissue of claim 1 wherein at least one pressure sensitive jaw closing mechanism adjusts electrosurgical energy delivery to tissue.

6. An electrosurgical instrument comprising a jaw structure configured to engage tissue, and a pressure sensitive variable resistive system within the instrument for adjusting electrosurgical energy delivery in response to tissue-engaging pressure.

7. The electrosurgical instrument of claim 6 wherein the pressure sensitive system comprises a jaw element having the capability of reversibly transforming from a substantially insulative state to a substantially conductive state under pressure.

8. The electrosurgical instrument of claim 6 wherein said jaw element is at least a portion of a jaw surface.

9. The electrosurgical instrument of claim 6 wherein said jaw element is interior of a jaw surface.

10. The electrosurgical instrument of claim 6 wherein the pressure sensitive system comprises a polymeric material capability of transforming from a substantially insulative state to a substantially conductive state under pressure.

11. The electrosurgical instrument of claim 10 wherein the polymeric material is a conductively doped elastomer.

12. The electrosurgical instrument of claim 10 wherein the substantially conductive state has an impedance of less than 500 ohms/cm.

13. The electrosurgical instrument of claim 10 wherein the substantially insulative state has an impedance of greater that 50 ohms/cm.

14. The electrosurgical instrument of claim 10 wherein the substantially insulative state has an impedance of greater that 10,000 ohms/cm.

15. The electrosurgical instrument of claim 10 wherein the substantially insulative state has an impedance of greater that 100,000 ohms/cm.

16. The electrosurgical instrument of claim 10 wherein the polymeric material transforms from a substantially insulative state to a substantially conductive state under a pressure ranging between 0.5 psi and 500 psi.

17. The electrosurgical instrument of claim 10 wherein the polymeric material transforms from a substantially insulative state to a substantially conductive state under a pressure ranging between 5 psi and 250 psi.

18. The electrosurgical instrument of claim 6 wherein the pressure sensitive system comprises a pressure sensitive variable resistive link in a jaw closing mechanism.

19. An electrosurgical instrument comprising a jaw structure configured to engage tissue, at least one jaw including a polymeric electrosurgical surface for applying electrosurgical energy to tissue, said at least one jaw includes an auxetic material for modifying a parameter or property of the electrosurgical surface.

20. The electrosurgical instrument of claim 19 wherein the auxetic material modifies compliant properties of the electrosurgical surface.
